(19) United States
(12) Patent Application Publication
(10) Pub. No.: US 2025/0177638 A1
(43) Pub. Date: Jun. 5, 2025

(54) MEMBRANE SEALS AND PROJECTIONS FOR MACROENCAPSULATION DEVICES

(71) Applicant: Vertex Pharmaceuticals Incorporated, Boston, MA (US)

(72) Inventors: Christopher Thanos, Providence, RI (US); Megan Billings, Warwick, RI (US); Matthew Watson, Stoneham, MA (US); Jason Robert Linden, Lincoln, RI (US); Kedar Chandrashekar Chitale, Little Rock, AR (US)

(73) Assignee: Vertex Pharmaceuticals Incorporated, Boston, MA (US)

(21) Appl. No.: 18/960,822

(22) Filed: Nov. 26, 2024

Related U.S. Application Data
(60) Provisional application No. 63/604,409, filed on Nov. 30, 2023.

(51) Int. Cl. A61M 5/14

(52) U.S. Cl. CPC A61M 5/14; A61M 2202/09; A61M 2207/00

(57) ABSTRACT

A macroencapsulation device includes a frame, a first membrane layer, a second membrane layer disposed on the first membrane layer, and an interior volume between the first and second membrane layers. The interior volume can encapsulate a population of cells. The frame includes a fill port extending from an exterior of the frame to an interior of the frame, with two slots formed in the frame on opposing sides of the fill port. The slots are formed on an interior portion of the frame. The first and second membrane layers are bonded together by a seal, which extends around and defines part of a perimeter of the interior volume. The seal includes two projections disposed in the two slots of the frame. A method of manufacturing includes bonding two membrane layers to form a seal defining a perimeter of an interior volume, and inserting the projections into the slots.

### CROSS-REFERENCE TO RELATED APPLICATIONS

This application claims priority under 35 U.S.C. § 119 (e) to U.S. Provisional Application Ser. No. 63/604,409, filed Nov. 30, 2023, and entitled “MEMBRANE SEALS AND PROJECTIONS FOR MACROENCAPSULATION DEVICES,” which is incorporated herein by reference in it its entirety for all purposes.

### FIELD

Disclosed embodiments are related to membrane seals and projections for macroencapsulation devices.

### BACKGROUND

Therapeutic devices that deliver biological products can be used to treat metabolic disorders, such as diabetes. The therapeutic devices may be implantable to provide a biological product, such as insulin, for an extended period of time. Some of these devices include macroencapsulation devices used to house cells to produce the desired biological product, a matrix including the cells, or other desired therapeutics within.

### SUMMARY

In some embodiments, a macroencapsulation device may comprise a frame including a fill port. The fill port may extend from an exterior of the frame to an interior of the frame. The frame may further include two slots extending into the frame on opposing sides of the fill port on an interior portion of the frame. The macroencapsulation device may further comprise a first membrane layer, and a second membrane layer disposed on the first membrane layer. An interior volume of the device may be disposed between the first membrane layer and the second membrane layer. The interior volume may be configured to encapsulate a population of cells. The first and second membrane layers may be bonded together by a seal extending around and defining at least a portion of the interior volume, and the seal may include two projections disposed within the two slots of the fame.

In other embodiments, a method of manufacturing a macroencapsulation device may include bonding a portion of a first membrane layer to a portion of a second membrane layer to form a seal extending around and defining at least a portion of a perimeter of an interior volume disposed between the first membrane layer and the second membrane layer. The method may further comprise inserting a first projection of the seal into a first slot of a frame of the macroencapsulation device, and inserting a second projection of the seal into a second slot of the frame. The first and second slots may be disposed on an interior portion of the frame on opposing sides of a fill port of the frame.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

In the drawings, each identical or nearly identical component that is illustrated in various figures may be represented by a like numeral. For purposes of clarity, not every component may be labeled in every drawing. In the drawings:

FIG. 1 is a top view of a macroencapsulation device, according to some embodiments;

FIG. 2 is a schematic top view of a fill port region of a macroencapsulation device in an unsealed configuration, according to some embodiments;

FIG. 3 is a schematic top view of a fill port region of a macroencapsulation device in an unsealed configuration, according to some embodiments;

FIG. 4 is a schematic top view of a fill port region of a macroencapsulation device in a scaled configuration with portions of the seal removed, according to some embodiments;

FIG. 5 is a cross-sectional side view of a macroencapsulation device, according to some embodiments;

FIG. 6 is a schematic top view of a fill port region of a macroencapsulation device in an unsealed configuration, according to some embodiments;

FIG. 7 is a flow chart depicting a method of manufacturing a macroencapsulation device, according to some embodiments.

### DETAILED DESCRIPTION

Macroencapsulation devices may be implanted in a patient to facilitate delivery and/or retention of therapeutics, biological products, cell populations, and/or other implantable materials. Such devices may be useful in the management and/or treatment of various diseases and conditions. As an illustrative example, management and/or treatment of diabetes may be facilitated by the implantation of a macroencapsulation device in which a population of insulin-producing cells is encapsulated. In this regard, the insulin-producing cells may be retained in the body to produce insulin within the body over a period of time.

Some macroencapsulation devices may include one or more membranes, which may form an interior volume in which implantable material(s) may be encapsulated. In some macroencapsulation devices, the membrane(s) may be configured to permit desired molecules, cells, and/or other matter to be transmitted through the membrane, while impeding the transmission of other molecules, cells, and/or other matter. Returning to the example of a macroencapsulation device for the treatment of diabetes, one or more membranes of such a device may be configured to allow oxygen, insulin, cell waste, and/or nutrients to pass through the membrane(s), while impeding the passage of immune cells. Such configurations may simultaneously facilitate survival of the insulin-producing cells and delivery of insulin from the cells to the patient. Accordingly, some embodiments may include treating a disease by implanting any of the macroencapsulation devices disclosed herein into a subject or patient in need thereof. In some such embodiments, the disease may be diabetes, although it will be appreciated that other diseases may additionally be suitable for treatment by such methods.

In some embodiments, the frame and/or a portion of the frame may include a fill port to facilitate the process of filling the interior volume with desired implantable material(s) (e.g., therapeutic(s), biological product(s), population(s) of cells, etc.). However, the inventors have appreciated that some macroencapsulation devices may experience issues related to interaction between the fill port, or a portion of the frame surrounding the fill port, and the seal formed in the membrane(s). In particular, cooperation between the seal and the fill port (or the frame surrounding the fill port) may impact the fluid-tight character, shape, and/or integrity of the interior volume and/or the one or more membranes. For example, gaps between the seal and the fill port/frame may cause discontinuities in the perimeter of the interior volume, allowing fluid to leak out of and/or into the interior volume. Additionally, the cooperation between the seal and the fill port/frame may impact the distribution of slack or excess material in the one or more membranes. For example, interference between the seal and the fill port/frame may create folds, wrinkles, and/or bunched material in the membrane(s). These may obstruct or otherwise interfere with a flow of fluid into or through the interior volume during a filling process. Accordingly, some devices may experience leaks, obstruction of flow through the fill port, incomplete fills, or other issues during and/or after a filling process as a result of undesirable interactions between the fill port (or other portion the frame surrounding the fill port) and the membrane seal.

In view of the above, the inventors have recognized and appreciated the benefits of a macroencapsulation device configured to form a fluid-tight closure at an interface between a scal formed in one or more membrane(s) forming an interior volume of a macroencapsulation device and a frame of the macroencapsulation device in an area surrounding a fill port. In some embodiments, a fluid-tight closure may be cooperatively formed by inserting a portion of the scal, such as a projection extending from an end portion of the seal towards a peripheral edge of the membrane(s) and into a correspondingly sized and shaped slot of the frame. In some embodiments, two projections may be inserted into two slots, with the slots disposed on opposing sides of a fill port to create a fluid-tight closure on either side of the fill port. In some embodiments, more than two projections may be inserted into more than two slots, for example to provide redundant closures (e.g., four or six projections may be inserted into four or six slots). The fluid-tight closure may be formed by creating fluid-tight contact between each slot and the respective projection, for example by applying heat and/or pressure to the slot and/or the surrounding region of the frame. In some embodiments, fluid-tight contact may be created by binding a projection within a slot, for example by plastically deforming, thermoplastically deforming, or otherwise causing material of the frame adjacent to the fill port to conform to conform to a size and shape of at least a portion of the projection of the seal adjacent to the fill port to form a fluid-tight interface with the projection. Alternatively, additional material, such an adhesive or binder may be used as an intermediate material between the frame and the projection of the seal to form a fluid tight seal between the slot formed in the frame and the projection of the seal disposed therein. In some embodiments, the slot and the projection may be formed with corresponding or complementary geometries to facilitate insertion of the projection into the slot, and/or to facilitate adequate and/or uniform contact between the slot and the projection. In some embodiments, the fill port region of the frame may be formed with excess material to ensure there is sufficient material to form the fluid-tight closure while maintaining the structural integrity of the frame.

In any of the embodiments of a macroencapsulation device disclosed herein, an interior volume may be formed in any appropriate manner and by any appropriate number of membranes. For example, an interior volume may be formed by two membranes, which may be bonded and/or sealed together around at least a portion of a perimeter of the interior volume to define at least the portion of the perimeter of the interior volume. In some embodiments, an interior volume may be enclosed by a single membrane extending around the entire interior volume. For example, the membrane may be bonded and/or sealed to itself, e.g., by folding the membrane back on itself. In some embodiments, a seal formed in the membrane(s) may extend around at least a portion of a perimeter of the interior volume to define the portion of the perimeter. In some embodiments, the seal may extend from a first end portion to a second end portion to partially define the perimeter of the interior volume, with a gap formed between the first and second end portions to permit fluid communication into/out of the interior volume. In some embodiments, each end portion may include a projection extending therefrom to at least partially define the gap. Some embodiments may include three membranes, four membranes, or any other appropriate number of membranes forming one interior volume, two interior volumes, three interior volumes, or any other appropriate number of interior volumes, as the disclosure is not particularly limited in this regard. Further, some macroencapsulation devices may include a frame to provide a desired structure, strength, shape, and/or rigidity to the device. For example, in embodiments where the membranes described above are flexible membranes, a rigid or semi-rigid frame may be included to provide the desired structure, strength, shape, and/or rigidity to the device. In some embodiments, the one or more membranes may be mounted to the frame. The frame may, in some cases, extend around at least a portion of a perimeter of the membrane(s), and/or a portion of a perimeter of the device.

In any of the embodiments of a macroencapsulation device or related methods disclosed herein, one or more membranes of a macroencapsulation device may comprise any appropriate porous biocompatible material. The porous biocompatible material may be substantially inert towards cells housed within the macroencapsulation device and the surrounding tissue. The biocompatible material may comprise a synthetic polymer, a naturally occurring polymer, glass fiber, cellulose, and/or any other appropriate biocompatible material or combination of materials. In some embodiments, the polymer may also be a linear polymer, a cross linked polymer, a network polymer, an addition polymer, a condensation polymer, an elastomer, a fibrous polymer, a thermoplastic polymer, a non-degradable polymer, combinations of the foregoing, and/or any other appropriate type of polymer as the disclosure is not limited in this fashion. As noted above, in one embodiment, a polymer may comprise expanded polytetrafluoroethylene (PTFE). Appropriate types of polymers may also comprise polyvinylchloride (PVC), polyethylene (PE), polycarbonate (PC), polyanhydrides, polyesters, polypropylene (PP), polymethylmethacrylate (PMMA), polystyrene (PS), polytetrafluoroethylene (PTFE), expanded polytetrafluoroethylene (ePTFE), polyurethane (PU), polyamide (nylon), polyethyleneterephthalate (PET), polyethersulfone (PES), polyetherimide (PEI), polyvinylidene difluoride (PVDF), polycaprolactone (PCL), poly(lactic-co-glycolic acid) (PLGA), poly-L-lactide (PLLA), polyacrylonitrile (PAN), electrospun PAN/PVC, polyetheretherketone (PEEK), any combination of the foregoing, and/or any other appropriate polymeric material. In some embodiments, a membrane used with any of the embodiments disclosed herein may comprise PVDF. In some embodiments, a membrane used with any of the embodiments disclosed herein may comprise electrospun PAN PVC. In some embodiments, a membrane used with any of the embodiments disclosed herein may comprise PES. In some embodiments, a membrane used with any of the embodiments disclosed herein may comprise PS. In some embodiments, a membrane used with any of the embodiments disclosed herein may comprise PAN. In some embodiments, a membrane used with any of the embodiments disclosed herein may comprise polycarbonate. In some embodiments, a membrane used with any of the embodiments disclosed herein may comprise polypropylene. In some embodiments, a membrane used with any one of the embodiments disclosed herein may comprise PVC. In some embodiments, a membrane used with any one of the embodiments disclosed herein may comprise PU. In some embodiments, a membrane used with any one of the embodiments disclosed herein may comprise PET. In some embodiments, a membrane used with any one of the embodiments disclosed herein may comprise PCL. In some embodiments, a membrane used with any one of the embodiments disclosed herein may comprise PLGA. In some embodiments, a membrane used with any one of the embodiments disclosed herein may comprise PLLA. In some embodiments, a membrane used with any one of the embodiments disclosed herein may comprise PMMA. In some embodiments, a membrane used with any one of the embodiments disclosed herein may comprise PEI. In some embodiments, a membrane used with any one of the embodiments disclosed herein may comprise nylon. In some embodiments, a membrane used with any one of the embodiments disclosed herein may comprise PTFE. In some embodiments, a membrane used with any one of the embodiments disclosed herein may comprise PE. The synthesis methods used for forming one or more of the porous membranes from the above noted polymeric materials may include, but are not limited to, expansion, solvent-casting, immersion precipitation and phase separation, electrospinning, methods that yield isoreticular networks, methods that yield trabecular networks, or any other appropriate method of forming a porous polymer membrane.

Sintering of a membrane may be used to alter the porosity and flux properties of a membrane. For example, the sintering may increase the porosity of the membrane while maintaining its pore structure. The sintering may also improve the mechanical stability and diffusive flux of the membrane. In some instances, a sintered membrane can have a lower melting temperature than an unsintered membrane of the same type. Further, sintered membranes may exhibit a different energy release during a differential scanning calorimetry scan, indicating a more relaxed structure in addition to the thickened porous network exhibited in sintered materials.

In view of the above, sintering may be used to alter the porosity and/or mechanical properties of the membranes, which in turn can be used to tune the porosity and the flux properties of the macroencapsulation device. Accordingly, in any of the embodiments described herein, any desired combination of sintered and/or unsintered membranes or membrane layers may be used. For instance, two exterior membrane layers of a device may be bonded together where either a sintered and unsintered membrane are bonded together, two sintered membranes are bonded together, or two unsintered membranes are bonded together. Further, any number of intermediate membranes positioned between these exterior membranes may be used where these intermediate membranes may be sintered or unsintered.

In any of the embodiments of a macroencapsulation device or related methods disclosed herein, adjacent membranes may be bonded to one another using an epoxy, a weld, or other fusion based technique (e.g., ultrasonic bonding, laser bonding, physical bonding, thermal bonding, etc.), mechanical interlocking, mechanical clamping using a frame or fixture, and/or any other appropriate bonding method. In some embodiments, adjacent membranes may be bonded using a heated tool that is used to press or strike two or more membranes against each other for a set fusion time with a predetermined pressure and/or force.

In any of the embodiments of a macroencapsulation device or related methods disclosed herein, the frame of a macroencapsulation device may be formed from any appropriate biocompatible thermoplastic material. As previously noted, in some embodiments, an appropriate material for the frame may include polyetheretherketone (PEEK). Appropriate materials for the frame may also include, but are not limited to polycarbonate (PC), polyurethane (PU), polyetheretherketone (PEEK), Polyvinyl Chloride (PVC), poly(oxymethylene), poly(methyl methacrylate) (PMMA), thermoplastic polymer based composites, polypropylene, fluorinated ethylene propylene (FEP), low density polyethylene (LDPE), high density polyethylene (HDPE), ultra-high density polyethylene (UHDPE), high molecular weight polyethylene (HMWPE), polystyrene (PS), polycaprolactone (PCL), poly(lactide), poly(glycolic acid) (PGA), poly lactide-co-glycolide, poly(lactic-co-glycolic acid) (PLGA), poly-L-lactide (PLLA), ethylene vinyl acetate copolymer, polyamides, poly(butylene) therephthalate, fluoropolymers (e.g., polytetrafluoroethylene (PTFE), polyvinylidene difluoride (PVDF), polyvinylpyrrolidone (PVP), and/or others), combinations of the foregoing, and/or any other appropriate thermoplastic material. In addition to the use of a thermoplastic material in a frame, embodiments in which a frame includes a thermoplastic portion configured to be bonded to a membrane and another non-thermoplastic portion are also contemplated as the disclosure is not limited to frames made completely from a thermoplastic material. In some embodiments, an appropriate material for the frame includes polypropylene. In some embodiments, an appropriate material for the frame includes fluorinated ethylene propylene (FEP). In some embodiments, an appropriate material for the frame includes ultra-high density polyethylene (UHDPE). In some embodiments, an appropriate material for the frame includes polycarbonate. In some embodiments, an appropriate material for the frame includes polyurethane. In some embodiments, an appropriate material for the frame includes PVC. In some embodiments, an appropriate material for the frame includes poly(oxymethylene). In some embodiments, an appropriate material for the frame includes poly(methyl methacrylate (PMMA). In some embodiments, an appropriate material for the frame includes thermoplastic polymer-based composites. In some embodiments, an appropriate material for the frame includes polypropylene. In some embodiments, an appropriate material for the frame includes LDPE. In some embodiments, an appropriate material for the frame includes HDPE. In some embodiments, an appropriate material for the frame includes polycaprolactone. In some embodiments, an appropriate material for the frame includes poly(lactide). In some embodiments, an appropriate material for the frame includes poly(glycolic acid). In some embodiments, an appropriate material for the frame includes poly lactide-co-glycolide. In some embodiments, an appropriate material for the frame includes ethylene vinyl acetate copolymer. In some embodiments, an appropriate material for the frame includes polyamides. In some embodiments, an appropriate material for the frame includes poly(butylene) therephthalate. In other embodiments, an appropriate material for the frame or portion of the frame may include titanium, graphene, stainless steel, or other appropriate biocompatible material exhibiting sufficient rigidity to function as a frame for the macroencapsulation device.

In any of the embodiments of a macroencapsulation device or related methods disclosed herein, a length of a slot in a frame, or slot length, may extend in a direction that is at least partially parallel, and in some instances substantially parallel, to a central axis of the slot. Additionally, a length of a slot may extend from an opening of the slot formed in an interior surface of the frame toward an exterior surface of the frame, for example along the central axis of the slot. In various embodiments, a slot length may be any appropriate length, as the disclosure is not particularly limited in this regard. In some embodiments, a slot length may be greater than or equal to 0.25 millimeters (mm), 0.50 mm, 0.75 mm, 1.00 mm, or any other appropriate length. Accordingly, in some embodiments, the slot length may be greater than or equal to 0.25 mm. In some embodiments, the slot length may be greater than or equal to 0.50 mm. In some embodiments, the slot length may be greater than or equal to 0.75 mm. In some embodiments, the slot length may be greater than or equal to 1.00 mm. Additionally or alternatively, in some embodiments, a slot length may be less than or equal to 2.00 mm, 1.50 mm, 1.25 mm, 1.00 mm, or any other appropriate length. Accordingly, in some embodiments a slot length may be less than or equal to 2.00 mm. In some embodiments a slot length may be less than or equal to 1.50 mm. In some embodiments a slot length may be less than or equal to 1.25 mm. In some embodiments a slot length may be less than or equal to 1.00 mm. Intermediate values between the foregoing are also contemplated, as are values both greater than and less than the foregoing. Combinations of the foregoing are also contemplated. For example, in some embodiments, a slot length may be greater than or equal to 0.25 mm and less than or equal to 2.00 mm. In some embodiments, a slot length may be greater than or equal to 0.50 mm and less than or equal to 1.50 mm. In some embodiments, a slot length may be greater than or equal to 1.00 mm and less than or equal to 1.25 mm. Ranges both greater than and less than the foregoing are also contemplated.

In any of the embodiments disclosed herein, a width of a slot in a frame, or slot width, may be a maximum width along a length and/or central axis of the slot, the slot width measured from one side of the slot to an opposing side of the slot. In various embodiments, a slot width may be any appropriate width, as the disclosure in not particularly limited in this regard. In some embodiments, a slot width may be greater than or equal to 0.25 millimeters (mm), 0.50 mm, 0.65 mm, 0.80 mm, or any other appropriate width. Additionally or alternatively, in some embodiments, a slot width may be less than or equal to 1.00 mm, 0.95 mm, 0.85 mm, 0.75 mm, or any other appropriate width. Accordingly, in some embodiments, a slot width may be less than or equal to 1.00 mm. In some embodiments, a slot width may be less than or equal to 0.95 mm. In some embodiments, a slot width may be less than or equal to 0.85 mm. In some embodiments, a slot width may be less than or equal to 0.75 mm. Intermediate values between the foregoing are also contemplated, as are values both greater than and less than the foregoing. Combinations of the foregoing are also contemplated. For example, in some embodiments, a slot length may be greater than or equal to 0.25 mm and less than or equal to 1.00 mm. In some embodiments, a slot length may be greater than or equal to 0.65 mm and less than or equal to 0.95 mm mm. In some embodiments, a slot length may be greater than or equal to 0.80 mm and less than or equal to 0.85 mm. Ranges both greater than and less than the foregoing are also contemplated.

In any of the embodiments of a macroencapsulation device or related methods disclosed herein, a slot may include a rounded portion. In some embodiments, the rounded portion may be a tip of the slot, which may be a portion of the slot that is furthest from the opening of the slot (for example, furthest along the length and/or the central of the slot from the opening in the interior surface of the frame, such that the tip and the opening are disposed at opposing ends of the length and/or central axis of the slot). In some embodiments, a rounded slot tip may facilitate more uniform contact between the frame material and the projection disposed in the slot, for example where the projection has a correspondingly rounded tip. Additionally or alternatively, the rounded tip may assist in maintaining a desired maximum internal stress in the frame, for example by reducing the likelihood and/or severity of stress risers (e.g., corners, discontinuities, etc.) in the frame material. In various embodiments, a radius of curvature of the tip of the slot may be any appropriate length, as the disclosure in not particularly limited in this regard. In some embodiments, a slot tip radius may be greater than or equal to 0.250 millimeters (mm), 0.300 mm, 0.350 mm, 0.400 mm, or any other appropriate length. Additionally or alternatively, in some embodiments, a slot tip radius may be less than or equal to 0.500 mm, 0.475 mm, 0.425 mm, 0.350 mm, or any other appropriate length. Intermediate values between the foregoing are also contemplated, as are values both greater than and less than the foregoing. Combinations of the foregoing are also contemplated. For example, in some embodiments, a slot tip radius may be greater than or equal to 0.250 mm and less than or equal to 0.500 mm. In some embodiments, a slot tip radius may be greater than or equal to 0.300 mm and less than or equal to 0.475 mm. In some embodiments, a slot tip radius may be greater than or equal to 0.350 mm and less than or equal to 0.425 mm. Ranges both greater than and less than the foregoing are also contemplated.

As noted above, a slot may extend in a direction from an inner surface of the frame located adjacent to the one or more membranes towards an exterior surface of the frame located radially outwards relative to the one or more membranes. Further, in any of the embodiments disclosed herein, the one or more slots may extend in a direction that is oriented at an angle relative to a direction in which a central axis of a fill port of the frame extends. Thus, in some embodiments, a slot may be substantially parallel (e.g., within 5° of parallel) to a central axis of the fill port (or the lumen of the fill port). In other embodiments, a slot may extend in a direction that is non-parallel to the fill port, and may be any appropriate angle with respect to a parallel angle. In some embodiments, a slot angle may be greater than or equal to 5°, 7.5°, 10°, 12.5°, or any other appropriate angle relative to the central axis of the fill port. Additionally or alternatively, in some embodiments, a slot angle may be less than or equal to 20°, 17.5°, 15°, 12.5° or any other appropriate angle relative to the central axis of the fill port. Intermediate values between the foregoing are also contemplated, as are values both greater than and less than the foregoing. Combinations of the foregoing are also contemplated. For example, in some embodiments, a slot angle may be greater than or equal to 5° and less than or equal to 20°. In some embodiments, a slot angle may be greater than or equal to 12.5° and less than or equal to 17.5°. Ranges both greater than and less than the foregoing are also contemplated.

In the present disclosure, the term “slot spacing” may refer to a spacing between two slots, and/or a spacing between two projections extending from end portions of a seal. A slot spacing may be measured between the central axes of two slots or projections at a point along the lengths of the slots/projections where the central axes of the two slots/projections are substantially parallel. In various embodiments, the slot spacing may be any appropriate spacing, as the disclosure in not particularly limited in this regard. In some embodiments, a slot spacing may be greater than or equal to 1.50 millimeters (mm), 2.00 mm, 2.25 mm, 2.50 mm, or any other appropriate spacing. Additionally or alternatively, in some embodiments, a slot spacing may be less than or equal to 4.00 mm, 3.25 mm, 3.00 mm, 2.75 mm, or any other appropriate spacing. Intermediate values between the foregoing are also contemplated, as are values both greater than and less than the foregoing. Combinations of the foregoing are also contemplated. For example, in some embodiments, a slot spacing may be greater than or equal to 1.50 mm and less than or equal to 4.00 mm. In some embodiments, a slot spacing may be greater than or equal to 2.00 mm and less than or equal to 3.25 mm. In some embodiments, a slot spacing may be greater than or equal to 2.50 mm and less than or equal to 2.75 mm. Ranges both greater than and less than the foregoing are also contemplated.

In any of the embodiments disclosed herein, a projection may be shaped and/or dimensioned such that the projection corresponds geometrically to a slot in which the projection is to be disposed. For example, a projection may be sized and shaped to be insertable into a slot before the slot is deformed/bonded to the projection as described herein. In this regard, the dimensions of a projection (e.g., a length, a width, a tip radius, and/or any other appropriate dimension) may be selected to correspond to the dimensions of a slot in a given embodiment. Of course, it will be appreciated that dimensions of a slot may conversely be selected to correspond to the dimensions of the projection, or that the dimensions of the slot and the dimensions of the projection may be selected cooperatively to achieve a desired geometric correspondence. In this regard, the disclosure is not limited to either one of the slot dimensions or the projection dimensions being selected based on the other. For example, in any of the embodiments described herein, a length, a width, and/or a tip radius may be any appropriate length, width, or radius, as described below.

In any of the embodiments disclosed herein, a length of a projection extending from an end portion of a seal, or projection length, may extend in a direction that is at least partially parallel, and in some instances substantially parallel, to a central axis of the projection. Additionally, a length and/or central axis of a projection may extend from an end portion of a seal. In various embodiments, a projection length may be any appropriate length, as the disclosure in not particularly limited in this regard. In some embodiments, a projection length may be greater than or equal to 2.00 millimeters (mm), 2.25 mm, 2.50 mm, or any other appropriate length. Additionally or alternatively, in some embodiments, a projection length may be less than or equal to 4.00 mm, 3.25 mm, 2.75 mm, 2.50 mm, or any other appropriate length. Intermediate values between the foregoing are also contemplated, as are values both greater than and less than the foregoing. Combinations of the foregoing are also contemplated. For example, in some embodiments, a projection length may be greater than or equal to 2.00 mm and less than or equal to 4.00 mm. In some embodiments, a projection length may be greater than or equal to 2.25 mm and less than or equal to 3.25 mm. In some embodiments, a projection length may be greater than or equal to 2.50 mm and less than or equal to 2.75 mm. Ranges both greater than and less than the foregoing are also contemplated.

In any of the embodiments disclosed herein, a width of a projection extending from an end portion of a seal, or projection width, may be a maximum width along a length and/or central axis of the projection, the projection width measured from one side of the projection to an opposing side of the projection. In various embodiments, a projection width may be any appropriate width, as the disclosure in not particularly limited in this regard. In some embodiments, a projection width may be greater than or equal to 0.25 millimeters (mm), 0.40 mm, 0.70 mm, or any other appropriate width. Additionally or alternatively, in some embodiments, a projection width may be less than or equal to 1.25 mm, 1.10 mm, 0.80 mm, or any other appropriate width. Intermediate values between the foregoing are also contemplated, as are values both greater than and less than the foregoing. Combinations of the foregoing are also contemplated. For example, in some embodiments, a projection length may be greater than or equal to 0.25 mm and less than or equal to 1.25 mm. In some embodiments, a projection length may be greater than or equal to 0.40 mm and less than or equal to 1.10 mm. In some embodiments, a projection length may be greater than or equal to 0.70 mm and less than or equal to 0.80 mm. Ranges both greater than and less than the foregoing are also contemplated.

In any of the embodiments disclosed herein, a tip radius of a projection extending from an end portion of a seal may be a radius of a rounded tip of the projection. The tip may be a portion of the projection that is furthest from the end portion of the seal from which the projection extends (for example, furthest along the length and/or the central of the projection from the end portion of the seal, such that the tip disposed at an opposing end of the length and/or central axis of the projection from a junction between the projection and the end portion of the seal). As noted above, a rounded tip of a projection and/or a rounded tip of a slot may facilitate uniform contact between the projection and the frame material and/or reduction of internal stress in the frame. In various embodiments, a radius of curvature of the tip of the projection may be any appropriate length, as the disclosure in not particularly limited in this regard. In some embodiments, a projection tip radius may be greater than or equal to 0.250 millimeters (mm), 0.300 mm, 0.350 mm, 0.370 mm, or any other appropriate length. Additionally or alternatively, in some embodiments, a projection tip radius may be less than or equal to 0.500 mm, 0.400 mm, 0.380 mm, 0.350 mm, or any other appropriate length. Intermediate values between the foregoing are also contemplated, as are values both greater than and less than the foregoing. Combinations of the foregoing are also contemplated. For example, in some embodiments, a projection tip radius may be greater than or equal to 0.250 mm and less than or equal to 0.500 mm. In some embodiments, a projection tip radius may be greater than or equal to 0.350 mm and less than or equal to 0.400 mm. In some embodiments, a projection tip radius may be greater than or equal to 0.370 mm and less than or equal to 0.380 mm. Ranges both greater than and less than the foregoing are also contemplated.

As noted above, fluid-tight contact may be created by binding a projection within a slot, for example by deforming material of the frame adjacent to the fill port to bring the frame material into fluid-tight contact with the projection, and/or by bonding the frame material to the projection material. The heat and/or pressure may raise the frame material to a melting temperature or glass transition temperature and cause the frame material to flow into contact with the projection. After the heat and pressure are removed, the cooled/depressurized material may then be in fluid-tight contact with the projection to create the desired fluid-tight closure. It will be appreciated that any appropriate method of forming the fluid-tight contact between the frame material and the projection of the seal may be used, including heat staking, hot welding, ultrasonic welding, cold welding (e.g., using pressure/force without applying heat), deforming the frame material, chemical bonding, adhesives, and/or any other appropriate method or combination of methods.

In any of the embodiments disclosed herein, a cell population contained within a compartment of a macroencapsulation device may be an insulin secreting cell population. In some embodiments, a cell population contained within a compartment of a macroencapsulation device comprises a heterogeneous population of cells. In some embodiments, the cell population comprises at least one cell derived from a stem cell derived cell. In some embodiments, at least one cell is a genetically modified cell. In some cases, at least one cell is genetically engineered to reduce an immune response in a subject upon implantation of the device, as compared to comparable cells that are not genetically engineered. In some embodiments, the cell population is a stem cell derived cell that is capable of glucose-stimulated insulin secretion (GSIS). For example, an appropriate population of cells may comprise pancreatic progenitor cells, endocrine cells, beta cells, a matrix including one or more of the foregoing, or any combination thereof. Further, a matrix may comprise isolated islet cells, isolated cells from pancreas, isolated cells from a tissue, stem cells, stem cell-derived cells (e.g., stem cell-derived islet cells), induced pluripotent cells, differentiated cells, transformed cells, or expression systems, which can synthesize one or more biological products. In some embodiments, the macroencapsulation device comprises a population of stem cell-derived islet cells. In some embodiments, the stem cell-derived islet cells comprise stem cell-derived beta cells, stem cell-derived alpha cells, and/or stem cell-derived delta cells.

Depending on the particular embodiment, a therapeutically effective density of cells may be loaded into one or more compartments of a macroencapsulation device. Appropriate cell densities disposed within a compartment may be greater than or equal to about 1,000 cells/μL, 10,000 cells/μL, 50,000 cells/μL, 100,000 cells/μL, 500,000 cells/μL, 750,000 cells/u L, 1,000,000 cells/μL, and/or any other appropriate cell density. Appropriate cell densities disposed within the compartment may also be less than or equal to about 1,000,000 cells/μL, 500,000 cells/μL, 100,000 cells/μL, 50,000 cells/μL, 10,000 cells/μL, and/or any other appropriate cell density. Combinations of the foregoing are contemplated including cell densities between about 1000 cells/μL and 1,000,000 cells/μL. In some embodiments, cell densities disposed within the compartment is between 100,000 cells/μL and 1,000,000 cells/μL. In some embodiments, cell densities disposed within the compartment is between 75,000 cells/μL and 500,000 cells/μL. In some embodiments, cell densities disposed within the compartment is between 500,000 cells/μL and 1,000,000 cells/μL. In some embodiments, cell densities disposed within the compartment is between 750,000 cells/μL and 1,000,000 cells/μL. In some embodiments, cell densities disposed within the compartment is between 750,000 cells/μL and 1,250,000 cells/μL. Of course, cell densities both greater than and less than those noted above may also be used depending on the desired application and cell types being used.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

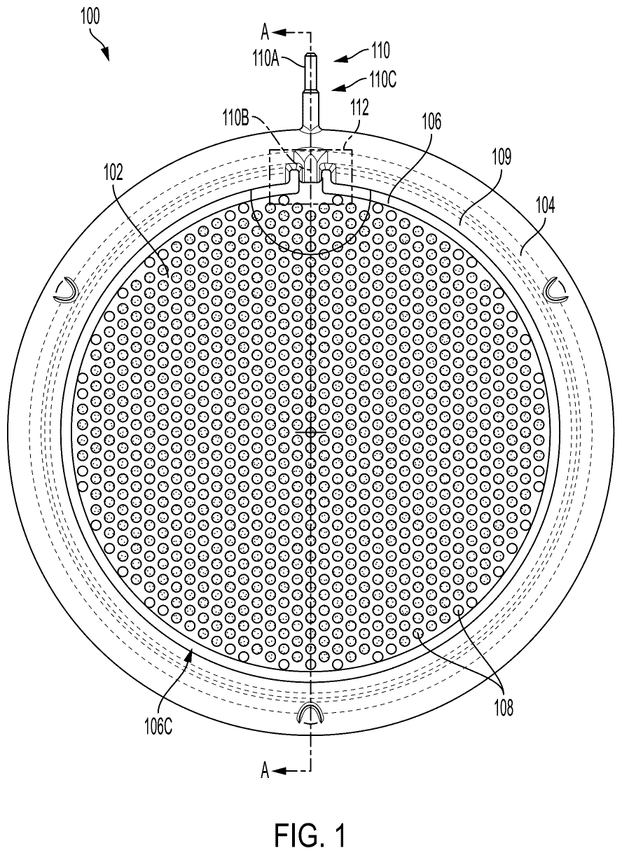

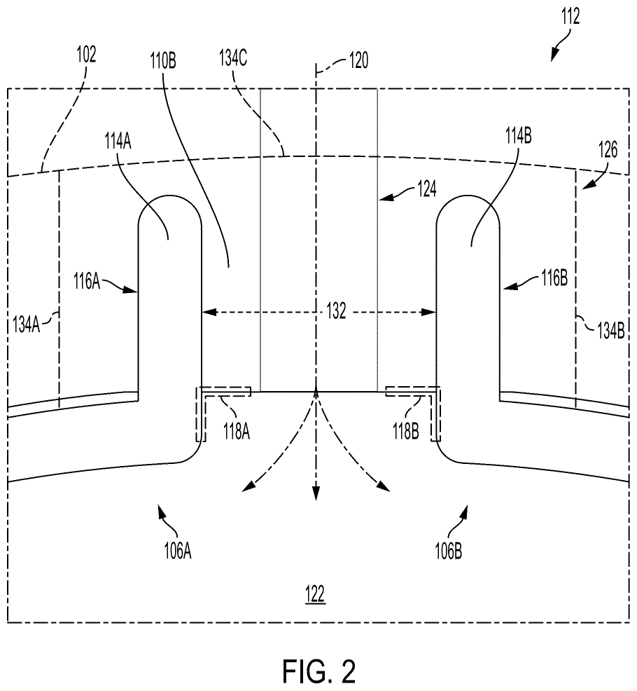

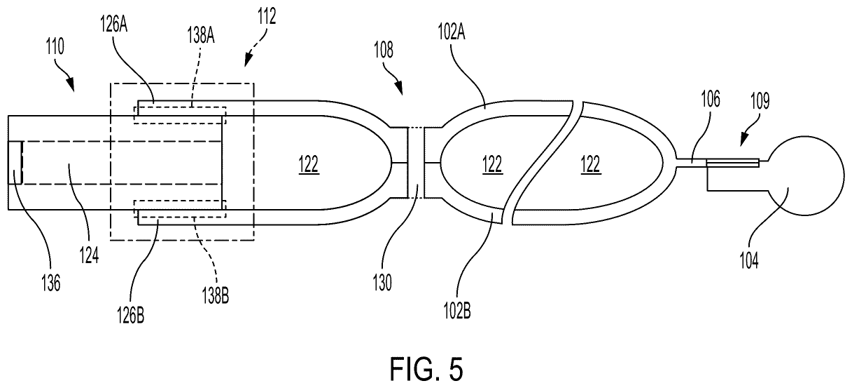

FIG. 1 depicts a macroencapsulation device 100 including one or more membranes 102 mounted to a frame 104. The one or more membranes include an interior volume configured to contain an implantable material (e.g., a population of cells, a therapeutic, a biological product, etc.). In this regard, the one or more membranes 102 may include a seal 106 extending around and defining at least a portion of a perimeter of the interior volume. In some embodiments, the scal may include first and second end portions 106A, 106B (see FIG. 2), as well as a first portion 106C extending between first and second end portions, with the first portion extending partially around a periphery of the membrane(s) 102, for example as shown in FIG. 1. In some embodiments, the seal 106 may be a bond line formed in the one or more membranes. The scal 106 may be formed by any appropriate method as described above. For example in some embodiments, a seal 106 may be formed by heat staking, hot welding, ultrasonic welding, cold welding (e.g., using pressure/force without applying heat), chemical bonding, adhesives, and/or any other appropriate method or combination of methods. Further to the above, in some embodiments, the one or more membranes may include one or more bonded portions 108 distributed across the interior volume of the one or more membranes and disposed radially inwards relative to seal 106 extending partially around the interior volume. As with the seal 106, the bonded portions 108 may be formed by any appropriate method, including heat staking, hot welding, ultrasonic welding, cold welding (e.g., using pressure/force without applying heat), chemical bonding, adhesives, and/or any other appropriate method or combination of methods. In some embodiments, at least a portion of the bonded portions 108 may include one or more through holes 130 extending from a first surface of the macroencapsulation device to a second opposing portions of the macroencapsulation device. These bonded portions 108 may be configured to maintain a desired surface-area-to-volume ratio of the device, maintain a desired maximum thickness of the device, and/or maintain a desired maximum distance for oxygen, nutrients, and/or cell waste to diffuse through the interior volume of the device to support survival of a cell population. As noted above, some of the bonded portions may include a through-hole 130 (see FIG. 5) which may help facilitate vascularization in a patient's body.

The one or more membranes 102 may be mounted to the frame 104 using any appropriate method, including heat staking, hot welding, ultrasonic welding, cold welding (e.g., using pressure/force without applying heat), deforming the frame material, mechanically interlocking portions of the membrane(s) with portions of the frame (e.g., by thermoforming the membrane(s) and the frame together), chemical bonding, adhesives, and/or any other appropriate method or combination of methods. Exemplary methods of mounting one or more membrane to a frame are described in PCT Application No. PCT/US2023/024510 (filed with the USPTO on Jun. 6, 2023), and in U.S. application Ser. No. 18/206,274 (filed with the USPTO on Jun. 6, 2023), both of which are incorporated herein by reference in their entireties. In some embodiments, the one or more membranes 102 may be mounted to the frame 104 at a frame/membrane interface region 109 of the device.

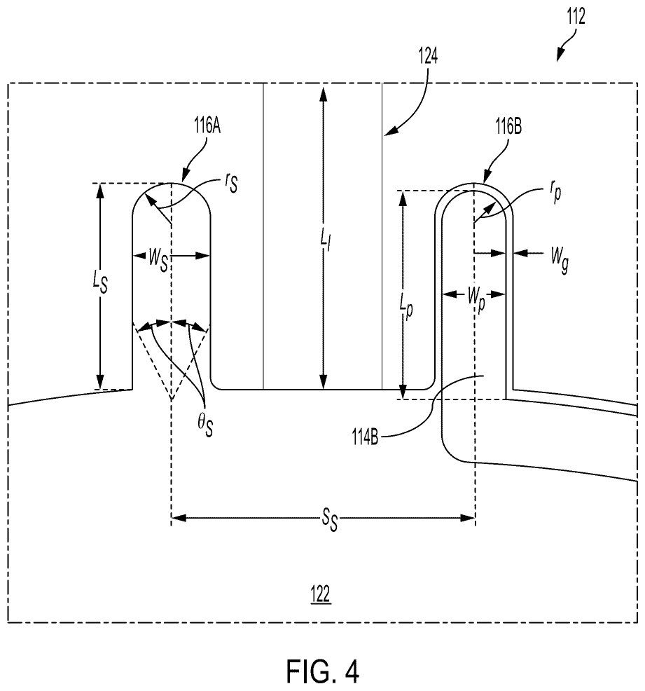

In some embodiments, a frame may include a fill port 110, which may extend from an exterior of the frame to an interior of the frame. For example, in the embodiment shown, the fill port 110 may include an exterior portion 110A and an interior portion 110B. As will be described further below, the fill port 110 may define a flow path extending through the frame from the exterior portion of the frame to the interior portion of the frame through which an implantable material (e.g., a therapeutic, biological product, population of cells, etc.) may be introduced into the interior volume of the one or more membranes 102. In this regard, the fill port 110 may be adjacent to and/or in contact with the membrane(s) 102 (e.g., to partially define the interior volume of the device), and may extend radially outwardly relative to the membrane(s) (e.g., to allow implantable material(s) to be introduced into the interior volume). In some embodiments, a fill port 110 may include or define a lumen extending from the exterior of the frame to the interior of the frame (see FIG. 4). For example, a lumen may extend through the fill port 110 from the exterior portion 110A to the interior portion 110B.

Additionally, a fill port 110 may be configured to engage with an injection system or other filling system to allow the implantable material(s) to be introduced into the interior volume through the fill port. For example, in the embodiment shown, the exterior portion 110A of the fill port may extend outwardly from an exterior of the frame and may be sized and shaped to form a fluid-tight engagement with a corresponding injection system (not shown). For example, the exterior portion 110A may be insertable into a portion of a filling system, and may be configured to facilitate flow of implantable material(s) into the device via the fill port, for example by forming a fluid-tight seal with the filling system. In some embodiments, an external diameter of the fill port may vary along a length of the fill port to facilitate forming a fluid-tight seal with a filling system. For example, the fill port 110 may include a step 110C and/or a tapered portion, at which an external diameter of the fill port narrows along a radial length of the fill port in a radially outward direction.

Further, the fill port 110 may have an open configuration in which the fill port is configured to permit flow of fluid through the fill port between an external environment and/or a filling system and the interior volume, and a closed configuration in which the fill port is configured to prevent fluid from flowing through the fill port between the interior volume and an external environment and/or filling system. In some embodiments, a fill port may be initially formed in an open configuration to allow implantable material(s) to be introduced to the interior volume via the open fill port. The fill port may subsequently be transitioned to a closed configuration to prevent fluid communication through the fill port, for example to enclose/encapsulate the implantable material(s) within the interior volume. In some embodiments, a lumen extending through the fill port may be open in the open configuration and closed in the closed configuration. In some embodiments, a fill port may be transitioned from the open configuration to the closed configuration by heating, melting, and/or thermoforming a portion of the fill port (for example, to close a lumen in either the exterior portion 110A, the interior portion 110B, or both). Additionally or alternatively, in some embodiments, an exterior portion of a fill port may be configured to be removed after the device has been filled, for example by cutting and/or melting the exterior portion. In some embodiments, transitioning the fill port from the open configuration to the closed configuration may include removing the exterior portion and closing of the fill port, either as separate steps or as a single step (e.g., using a heated cutting tool to simultaneously cut off the exterior portion and melt a resulting cut face to close the fill port). Further, it will be appreciated that a fill port may be closed by any appropriate method, including by welding a lumen or aperture of the fill port closed, by pinching the fill port closed, by filling the lumen or aperture (e.g., with curable material, a sealant, a filler, a plug, etc.), and/or by any other appropriate method or combination of methods.

In some embodiments, a perimeter of the interior volume may be at least partially defined by the seal 106, and at least partially defined by the frame or a portion of the frame (e.g., a fill port in a closed configuration). In the embodiment shown, the seal 106 may cooperate with a fill port region 112 of the frame to at least partially define the interior volume. The fill port region 112 may be a portion of the frame which is near or adjacent to a fill port, and may include any appropriate portion of the frame, including internal and/or external portions of the frame. In the embodiment shown, the fill port region 112 may include the internal portion 110B of the fill port 110, as well as other internal portions of the frame near the fill port such as a portion of the frame/membrane interface region 109.

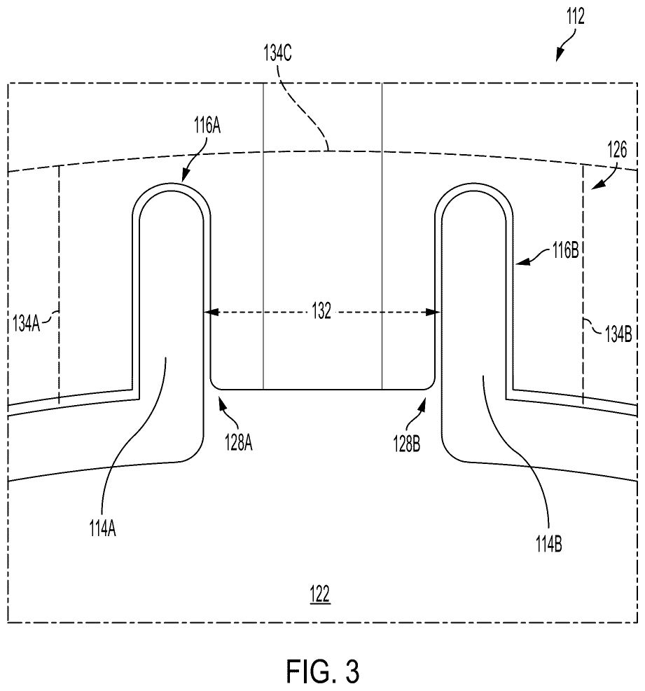

A close-up view of a fill port region 112 in a sealed configuration according to some embodiments is shown in FIG. 2. As explained further below, in the sealed configuration, the fill port region 112 may include one or more fluid-tight closures formed between a seal of the membrane(s) and a portion of the frame, for example at first and second interfaces 118A, 118B. As will be appreciated by comparison with FIG. 3, a fill port region in an unscaled configuration may include gaps 128A, 128B instead of the fluid-tight closures at the interfaces 118A, 118B. A distinction should be appreciated between: (a) the sealed and unsealed configurations of the fill port region, and (b) the open and closed configurations of the fill port itself. In this regard, a fill port region may be in a sealed configuration (e.g., may include a fluid-tight closure between a seal and the frame) even though the fill port is in an open configuration (e.g., to permit fluid to flow into or out of the interior volume). For example, a device may be formed with a fill port in an open configuration and a fill port region in an unsealed configuration (e.g., FIG. 3). The fill port region may be transitioned from the unscaled configuration to a sealed configuration (e.g., by bonding the frame and membranes together as shown in FIG. 2) while the fill port remains in the open configuration. This may prepare the device to receive implantable material(s) in the interior volume. Implantable material(s) may be introduced into the interior volume while the fill port is in the open configuration and the fill port region is in the sealed configuration. The fill port may be transitioned from the open configuration to the closed configuration while the implantable material(s) is/are disposed in the interior volume, and while the fill port region is in the sealed configuration. This may encapsulate the implantable material(s) within the interior volume.

As shown in FIG. 2, the fill port region 112 may include an interior portion 110B of a fill port 110, as well as portions of the seal 106 such as end portions 106A, 106B of the seal 106. In some embodiments, one or more portions of the seal may be configured to contact and/or be disposed within the frame or a portion of the frame. For example, the seal 106 may include two projections, such as a first projection 114A and a second projection 114B. In some embodiments, the projections 114A, 114B may extend from respective end portions 106A, 106B of the scal 106.

Additionally, in some embodiments, the frame (or a portion of the frame) may be configured to contact and/or receive a portion of the seal. For example, an interior portion of the frame 104 may include two slots, such as first slot 116A and second slot 116B. As shown in FIG. 2, each projection 114A, 114B of the seal may be disposed within a respective slot 116A, 116B of the frame. In this regard, each slot 116A, 116B may be sized and shaped to receive a respective projection 114A, 114B, and each projection 114A, 114B may be sized and shaped to be inserted into a respective slot 116A, 116B. In some embodiments, each projection extending from the seal may cooperate with a respective slot of the frame to form a fluid-tight closure. For example, the first projection 114A may cooperate with the first slot 116A to form a fluid-tight closure at a first interface 118A, and the second projection 114B may cooperate with the second slot 116B to form a fluid-tight closure at a second interface 118B. As will be described in detail below, in some embodiments, a fluid-tight closure may be formed by heating the fill port region and thermoforming the slots 116A, 116B to seal against the projections 114A, 114B, although other ways to form a fluid-tight closure are also within the scope of this disclosure. In some embodiments, each slot may be in contact with the respective projection around at least a portion of a perimeter of the respective projection to prevent fluid from flowing through the slot around the perimeter of the respective projection.

An opening 132 may be defined by the projections 114A, 114B, and may be configured to permit fluid communication between the interior volume 122 and the fill port 110. The opening 132 may allow implantable material(s) to be introduced into the interior volume across the scal 106 via the fill port 110 by permitting flow between the projections 114A, 114B and/or between the end portions 106A, 106B of the seal. Additionally, the one or more membranes may be bonded to the frame across the opening 132. In some embodiments, as will be described further with reference to FIG. 5, each of a first membrane layer and a second membrane layer may be bonded to a respective side of the fill port to partially define the interior volume.

As noted above, the fill port may define a flow path configured to permit implantable material(s) to be injected or otherwise introduced from an externally located filling system into the interior volume of the device. For example, the fill port 110 may define a flow path 120 extending from the exterior portion 110A to the interior portion 110B through the frame to permit implantable material(s) to flow into the interior volume 122. In some embodiments, the flow path 120 may extend between the two projections 114A, 114B (e.g., through the opening 132) and into the interior volume 122. Additionally, in some embodiments, the flow path may be formed by a lumen 124 of the fill port 110, which may extend through the fill port from an exterior portion to an interior portion of the fill port (e.g., from an exterior aperture of the fill port to an interior aperture of the fill port).

As will be explained in further detail below with reference to FIG. 5, the membrane(s) 102 may include one or more flaps to facilitate mounting the membrane(s) to the frame in the fill port region while permitting fluid communication into the interior volume. In some embodiments, the membrane(s) 102 may be cut or otherwise separated on opposing sides of the projections 114A, 114B in order to form a flap 126. In some embodiments, a perimeter of the flap 126 may extend at least partially around both the first projection 114A and the second projection 114B. For example, the perimeter of each flap 126A, 126B may include a first cut perimeter 134A adjacent to the first projection 114A on an opposing side of the first projection from the opening 132, and a second cut perimeter 134B adjacent to the second projections 114B on an opposing side of the second projection 114B from the opening 132. A perimeter of the overall membrane (e.g., membrane perimeter 134C) may join the first and second cut perimeters to form the flap. Of course, it will be appreciated that although the depicted flaps include two cut perimeters joined by a perimeter of the membrane, other flap geometries are also contemplated as the disclosure is not particularly limited in this regard. For example, a flap may be formed by cutting a single continuous cut perimeter into the membrane, or a membrane may be formed or provided with an overall membrane perimeter in a shape that includes a flap. Additionally, in embodiments which include more than one membrane layer, each membrane layer may include a respective flap. In some such embodiments, each flap may be formed in substantially the same shape having substantially a similar perimeter (e.g., the perimeter of flap 126). However, embodiments in which two or more flaps are shaped differently and/or are not coextensive with one another are also contemplated. Additionally, it will be appreciated that some embodiments, including embodiments with more than one membrane layer, may include only one flap formed in only one membrane layer, as even a single flap may facilitate a spaced relationship between the membrane layers, as discussed with reference to FIG. 5 below. Furthermore, embodiments in which no flaps are included in any membrane layers (e.g., where each membrane layer is contiguous) are also contemplated.

FIG. 3 depicts a fill port region 112 in an unsealed configuration (e.g., during a manufacturing process) and with the fill port in an open configuration in which material may flow through the lumen of the fill port. In the unsealed configuration, one or more gaps may exist between a seal of the membrane(s) and the frame, for example gaps 128A, 128B disposed between the seal projections 114B and the associated slot 116B. In the embodiment shown, the slots 116A, 116B and/or projections 114A, 114B may be sized and shaped such that there are one or more gaps 128A, 128B between the slots and seal projections when the seal projections are inserted into the slots. In some embodiments, fluid-tight closures between the projections 114A, 114B and the slots 116A, 116B may be formed by filling, closing, or otherwise sealing the gaps 128 during a manufacturing process. For example, material from the frame 104 in the fill port region 112 may be used to fill the gaps 128A, 128B to form a seal between the seal projections and the frame. In some embodiments, the frame 104 may be formed from a thermoplastic material (e.g., PEEK or others), such that application of heat and/or pressure to the fill port region may allow material from the frame 104 to deform or flow into contact with the projections of the end portions of the seal disposed therein, thereby filling the gap to form a fluid-tight closure. For example, the frame may be heated to a suitable temperature greater than a glass transition and/or melting temperature of the frame material. Additionally or alternatively, pressure may be applied to the frame, the fill port region, and/or a slot to facilitate flow and/or deformation of the frame material to form the fluid-tight closure. In either case, the frame and/or scal projections may undergo plastic deformation using a press, striker, or other appropriate thermoplastic bonding and/or formation process, though embodiments in which a sealant, adhesive, or other material is added to fill the slots are also contemplated. In some embodiments, the frame material surrounding the slot may be flowed into contact with a projection of the seal around at least a portion of a perimeter of the projection forming an intimate interface that corresponds to a size and shape of the corresponding portion of the projection of the seal disposed in the slot such that fluid may be prevented from flowing through the slot around the perimeter of the projection of the seal of the one or more membranes (e.g., through the gaps 128A, 128B).

FIG. 4 depicts the arrangement of FIG. 3, with the first end portion 106A of the scal and the first projection 114A removed to more clearly illustrate various features of the fill port region 112. In some embodiments, a slot may be formed as an elongated opening extending through the frame in a first direction (e.g., from a first surface of the frame to a second surface opposite the first surface in a direction into the page as shown in FIG. 4), and extending into the frame in a second direction (e.g., from an opening formed in an interior surface of the frame towards an exterior surface of the frame). For example, the first slot 116A may include an opening formed in an interior surface of the frame at an open end portion, and may extend from the opening into the fame in a direction substantially parallel to a plane in which the membrane(s) and/or interior volume extends within the frame. In some embodiments, a slot may extend into the frame by a slot length Ls extending along a central axis of the slot, and may have a slot width Wₛ. Additionally, in some embodiments, a slot may terminate in a tip that is oriented approximately outward away from the interior volume formed by the bonded layers of the one or more associated membranes mounted in the frame. It should be understood that the tip at the outer end portion of the slot may have any appropriate geometry. In the depicted embodiment, the slots 116A, 116B may each terminate in a rounded tip having a radius of curvature rₛ. In some embodiments, two slots may be formed on opposing sides of a fill port. In some such embodiments, the slots may be spaced apart by a slot spacing Sₛ, with the slot spacing being measured as a distance between the central axes of two adjacent slots. In some embodiments, two slots may be disposed symmetrically on either side of a fill port, such that a central axis of each slot is disposed at the same distance from a central axis of the fill port (or lumen of the fill port). In other embodiments, two slots may be disposed and/or oriented asymmetrically, as the disclosure is not particularly limited in this regard.

In some embodiments, a slot and a projection extending from an end portion of a scal may have corresponding geometries. For example, a projection may have a projection length Lₚ extending along a central axis of the projection, and a projection width Wₚ. Additionally or alternatively, a projection may terminate in a tip, which may have any appropriate geometry. In the depicted embodiment, the projections 114A, 114B may each terminate in a rounded tip having a radius of curvature rₚ. In various embodiments, the slot length Ls may be selected to correspond to the projection length Lₚ; the slot width Wₛ may be selected to correspond to the projection width Wₚ; and/or the slot tip radius of curvature rₛ may be selected to correspond to the projection tip radius of curvature rₚ. In this regard, correspondence may signify that two corresponding values are about equal, or correspondence may signify any other appropriate relationship between the two corresponding values. For example, the slot dimensions may be selected to correspond to the projection dimensions to create a gap between the slot and the projection when the projection is disposed within the slot. In some embodiments, the relative dimensions of the slot and the projection may be selected to correspond such that a desired gap width W_{g} may be achieved between the slot and the projection when the projection is disposed within the slot.

Further to the above, a slot and/or a projection extending from an end portion of a scal may be formed with a desired spatial relationship to other parts of a macroencapsulation device. In some embodiments, a slot and/or a projection may be formed to be substantially parallel to a fill port of the device. For example, a slot length Ls and/or a projection length Lₚ may extend substantially parallel to an axial length of the fill port, or to a lumen length Li extending along a central axis of a lumen 124 through the fill port. In this regard, two lengths may be substantially parallel when there is a deviation of 5° or less between the orientations of the two lengths. In other embodiments, a slot may be formed having a slot angle θₛ, with the slot angle being measured as a deviation in an orientation of the slot from an orientation of the lumen or other reference feature of the fill port. The slot angle θₛ may be selected such that a slot is angled towards the fill port or away from the fill port, and the slot angles for the two opposing slots disposed on either side of the fill port may be the same or different.

FIG. 5 depicts a cross-sectional view of a macroencapsulation device according to some embodiments, for example as taken along the line A-A of FIG. 1. In some embodiments, the one or more membranes may include a first membrane layer 102A and a second membrane layer 102B disposed on the first membrane layer. In various embodiments, the first and second membrane layers 102A, 102B may be two separate membranes, or may be a single membrane (e.g., where a single membrane has been folded back on itself to form multiple layers. As noted above, each of the first and second membrane layers may be formed from any appropriate material, including a sintered porous biocompatible polymer and/or an unsintered porous biocompatible polymer. In some embodiments, the first membrane layer may be a sintered porous biocompatible polymer (e.g., sintered ePTFE or others), and the second membrane layer may be an unsintered porous biocompatible polymer (e.g., unsintered ePTFE or others), though embodiments in which both membrane layers are sintered are also contemplated.

The first and second membrane layers may be bonded and/or fused together at various locations, for example to form the seal 106 extending around a portion of the interior volume 122 of the macroencapsulation device and/or the bonded portions 108 disposed inwards from this outer seal. As noted above, some bonded portions 108 may optionally include a through hole 130 to facilitate vascularization of the device upon implantation. In some embodiments, the scal 106 may at least partially define a perimeter of the interior volume 122. Additionally, in some embodiments, the first and second membrane layers 102A, 102B may be mounted to the frame 104 in a frame/membrane interface region 109. In some embodiments and as shown in the figure, the first and second membrane layers 102A, 102B may be disposed in a stacked configuration in the frame/membrane interface region, such that the membrane layers are in direct contact with one another. In some embodiments, this stacked configuration may facilitate the processes of scaling the membrane(s), mounting of the membrane layers to the frame, and/or other manufacturing processes. However, embodiments in which one or more intermediate layers, coatings, or other structures are positioned between the membrane layers and/or between the membranes and the frame are also contemplated as the disclosure is not so limited.

In some embodiments and as will be appreciated with reference to FIGS. 1-3, the seal 106 may be discontinuous in the fill port region 112 such that the protrusions 114A and 114B located on the separate end portions of the seal 106 and extending out from the interior volume 122 towards an outer perimeter of the membrane layers 102A and 102B may form an opening 132 defined between the opposing protrusions 114A and 114B of the seal 106 and the opposing layers of membrane 102A and 102B. The opening 132 may be fluidly connected to the interior volume of the scaled membrane layers as described above and may be used to introduce a desired material, such as one or more cell populations, across the sealed perimeter of the interior volume 122 and into the interior volume 122 via the fill port 110. Additionally, it will be appreciated that a stacked configuration of the membrane layers 102A, 102B, in which the membrane layers are in direct contact, may prevent or impede implantable material(s) from being introduced to the interior volume 122, for example by blocking fluid communication through the fill port 110 to the interior volume 122. Accordingly, in some embodiments, the first membrane layer 102A and the second membrane layer 102B may be spaced apart in the fill port region to avoid blocking of the above noted opening. For example, in some embodiments, the first and second membrane layers may be disposed on opposing respective sides of the fill port 110. In some embodiments, the separation of the membrane layers may be facilitated by forming one or more flaps in one or more of the membrane layers in the area of the fill port region. For example, a first flap 126A may be formed in the first membrane layer where the first flap 126A is disposed on a first side of the fill port 110 when assembled with the frame.

Each flap and/or membrane layer may be bonded to the frame in the fill port region to form a scal between the membrane layer and the frame. For example, each flap be bonded to a respective side of the frame in a respective fill port bond area 138A, 138B. In some embodiments, each fill port bond area 138A, 138B may extend across the width of the opening 132 (see FIGS. 2-3), for example from the first projection 114A to the second projection 114B, to facilitate containment of the implantable material(s) within the interior volume 122. In various embodiments, a flap may be bonded to a frame using any appropriate method, including methods similar to those used to bond the membrane layer(s) to the frame 104 (e.g., at the frame/membrane interface 109). For example, a flap may be bonded to the frame using heat staking, hot welding, ultrasonic welding, cold welding (e.g., using pressure/force without applying heat), deforming the frame material, mechanically interlocking the membrane to the frame, chemical bonding, adhesives, and/or any other appropriate method or combination of methods. As will be appreciated, the arrangement shown in FIG. 5 may facilitate the encapsulation of the interior volume 122, and any implantable material(s) disposed therein (e.g., population(s) of cells, therapeutic(s), biological product(s), etc.). In particular, when the fill port 110 is closed (e.g., by cutting, melting, filling, pinching, and/or otherwise closing the exterior portion as described above, thereby closing the lumen 124 as shown by the lumen closure 136), the interior volume may be cooperatively encapsulated and/or defined by the first membrane layer 102A, the second membrane layer 102B, the seal 106, the fluid-tight closures formed between the slots and the projections disclosed herein (e.g., at the interfaces 118A, 118B), the fill port bond areas 138, 138B, and the lumen closure 136. It will further be appreciated that although the lumen closure 136 is illustrated as closing an exterior aperture of the lumen 124, other configurations in which the fill port/lumen is closed at an interior aperture, or at any intermediate location between the exterior aperture and the interior aperture are also contemplated, as the disclosure is not limited in regard to where a fill port or lumen may be closed. For example, in some embodiments, an interior of the lumen may be closed concurrently with the formation of the fluid-tight closures between the slots and frames described herein, such that a single application of heat and/or pressure may close the fill port while also forming the fluid-tight closures.

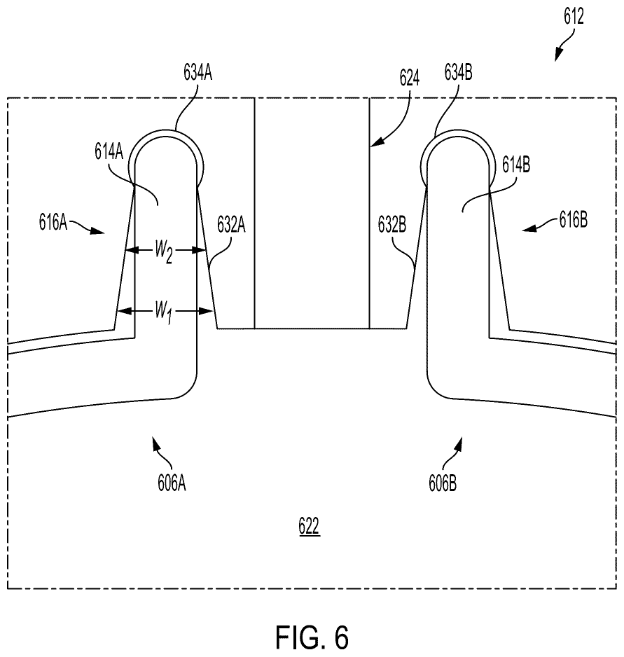

FIG. 6 illustrates that the slots 616A, 616B may be formed in any appropriate shape to receive the projections 614A, 614B in the fill port region 612. For example, each slot 616A, 616B may include a tapered portion 632A, 632B and/or a rounded portion (e.g., rounded tips 634A, 634B). In some embodiments, each tapered portion may be configured to facilitate insertion and/or positioning of a projection in the respective slot. For example, a tapered portion may guide the projection towards the rounded tip during insertion. In some embodiments, each tapered portion may be formed as an opening having a first width W₁ at first point along a length of the opening and a second width W₂ at a second point along the length of the opening. In some embodiments, the first point may be at an interior location relative to the second point (i.e., closer to a center of the device than the second point), and the first width may be greater than the second width. Further, in some embodiments, the rounded portion may be disposed at a narrower end of the tapered portion, such that the rounded portion and tapered portion cooperatively form the slot into a “keyhole” shape, as shown in FIG. 6.

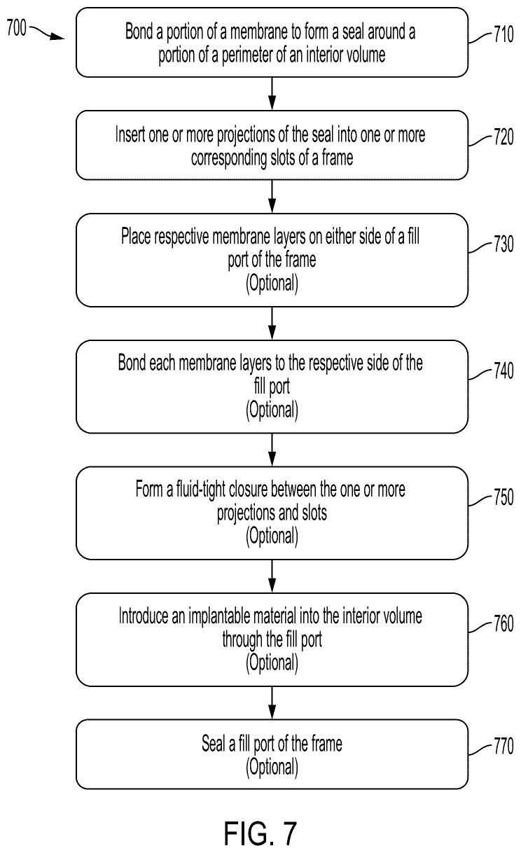

FIG. 7 is a flow chart illustrating a method 700 for manufacturing a macroencapsulation device according to some embodiments. At step 710, a portion of one or more membranes may be bonded to form a seal extending around and defining at least a portion of a perimeter of an interior volume in the one or more membranes. In various embodiments, the one or more membranes may be bonded by heat staking, hot welding, ultrasonic welding, cold welding (e.g., using pressure/force without applying heat), deforming the frame material, chemical bonding, adhesives, and/or any other appropriate method or combination of methods.

At step 720, a first projection may be inserted into a first slot of the frame. In some embodiments, a second projection may additionally be inserted into a second slot of the frame. The first and second projections may extend from respective end portions of a seal formed in the membrane(s). The first and second slots of the frame may be disposed on an interior portion of the frame on opposing sides of a fill port of the frame, as disclosed herein.

At step 730, a flap may be cut into at least one of the one or more membranes, and the flap may be placed on a side of the frame, or on a side of a fill port of the frame. For example, a first flap may be cut into a first membrane layer and/or a second flap may be cut into a second membrane layer. The first flap may be placed on a first side of the frame, and/or the second flap may be placed on a second side of the frame opposite the first side. Furthermore, the first and second flaps may be placed on opposing sides of a fill port of the frame. As noted above, in some embodiments, only a first flap may be cut in the first membrane layer, and the first flap may be placed on a first side of the fill port opposite a second side of the fill port on which the second membrane layer may be placed.

At step 740, each membrane layer may be bonded to the respective side of the frame, or to the respective side of the fill port. In some embodiments, this may include bonding and/or scaling each membrane layer (or flap thereof) to the respective side of the fill port. For example, in some embodiments, each membrane layer (or flap thereof) may be bonded to the respective side of the fill port across a width of an opening defined by the projections. In some embodiments, this may prevent fluid communication through the opening between the membrane layer (or flap) and the respective side of the fill port. In some embodiments, each membrane layer may be bonded to the respective side of the fill port by heat staking, hot welding, ultrasonic welding, cold welding (e.g., using pressure/force without applying heat), deforming the frame material, mechanically interlocking the membrane/flap to the frame, chemical bonding, adhesives, and/or any other appropriate method or combination of methods. In some embodiments, each membrane layer may be bonded to a respective side of the frame as described in PCT Application No. PCT/US2023/024510, and in U.S. application Ser. No. 18/206,274, both of which, as noted above, are incorporated herein by reference in their entireties.

At step 750, a fluid-tight closure may be formed between the projection and the slot, or between each projection and each respective slot (e.g., forming a first fluid-tight closure between the first projection and the first slot, and forming a second fluid-tight closure between the second projections and the second slot). In some embodiments, a fluid-tight closure may be formed by contacting each projection with the respective slot around at least a portion of a perimeter of the respective projection to prevent fluid from flowing through the slot around the perimeter of the respective projection. For example, in some embodiments, each projection may be intimately mated with a respective slot by heating the slot (and/or the fill port region) to a temperature greater than a melting and/or a glass transition temperature of the frame, applying a pressure to the frame with an appropriate die, striker, ultrasonic horn, and/or other molding tool, and flowing material of the slot such that the slot conforms to a size and shape of the associated projection of the seal of the membranes disposed within the slot to form a seal therebetween. In some embodiments, the fluid-tight closure may be formed by heat staking, hot welding, ultrasonic welding, cold welding (e.g., using pressure/force without applying heat), deforming the frame material, chemical bonding, adhesives, and/or any other appropriate method or combination of methods.

At step 760, one or more implantable materials may be introduced into an interior volume of the device. For example, one or more therapeutics, biological products, and/or populations of cells may be introduced into the interior volume. In some embodiments, one or more population of cells may include a population of insulin-producing cells. In some embodiments, a population of cells may be injected into the interior volume via a fill port. Additionally, in some such embodiments, the population of cells may be injected by an external injection system via the fill port.

At step 770, the fill port may be sealed. In some embodiments, sealing the fill port may fully encapsulate the interior volume of the device. Additionally, in some embodiments, scaling the fill port may encapsulate at least one population of cells in the interior volume. Furthermore, in some embodiments, sealing a fill port may comprise heating the fill port to a melting temperature or a glass transition temperature of the fill port, and/or applying pressure to the fill port. Further, in some embodiments, scaling a fill port may include closing a lumen of the fill port, welding a lumen or aperture of the fill port closed, by pinching the fill port closed, by filling the lumen or aperture (e.g., with curable material, a sealant, a filler, a plug, etc.), or otherwise scaling the lumen of the fill port in any other appropriate manner as the disclosure is not so limited.

Although the depicted embodiments illustrate two projections and two slots, additional projections and additional slots may also be included. For example, third and fourth projections may cooperate and/or bond with third and fourth slots to provide redundant fluid-tight closures. Such additional slots and projections may be formed at any appropriate location(s), including locations within or outside of the fill port region. Furthermore, any additional projections may extend from any appropriate location or portion of a seal, including portions located away from the end portions at which the first and second projections are depicted in the figures (e.g., the first portion 106C, as shown in FIG. 1). Accordingly, it will be appreciated that a macroencapsulation device may include any appropriate number of projections and slots forming any appropriate number of fluid-tight closures as described herein, and that a method of manufacturing a macroencapsulation device may include forming any appropriate number of projections and slots to form any appropriate number of fluid-tight closures as described herein.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A macroencapsulation device, comprising:
a frame including a fill port extending from an exterior of the frame to an interior of the frame, and two slots extending into the frame on opposing sides of the fill port on an interior portion of the frame;
a first membrane layer;
a second membrane layer disposed on the first membrane layer; and
an interior volume disposed between the first membrane layer and the second membrane layer, the interior volume configured to encapsulate a population of cells,
wherein the first and second membrane layers are bonded together by a seal extending around and defining at least a portion of a perimeter of the interior volume, the seal including two projections disposed within the two slots of the frame.

2. The macroencapsulation device of claim 1, further comprising a flow path extending through the fill port between the two projections and into the interior volume.

3. The macroencapsulation device of claim 1, wherein a first portion of the seal extends partially around a periphery of at least one of the first membrane layer and the second membrane layer, the first portion extending from a first end portion of the seal to a second end portion of the seal.

4. The macroencapsulation device of claim 3, wherein each projection of the two projections extends from a respective end portion of the first and second end portions towards a peripheral edge of the at least one membrane.

5. The macroencapsulation device of claim 1, wherein the first membrane layer includes a first flap disposed on a first side of the frame.

6. The macroencapsulation device of claim 5, wherein the second membrane layer includes a second flap disposed on a second side of the frame opposite the first side.

7. The macroencapsulation device of claim 6, wherein the first flap is on a first side of the fill port of the frame and the second flap is on a second side of the fill port opposite the first side of the fill port.

8. The macroencapsulation device of claim 1, wherein each projection cooperatively forms a fluid-tight closure with the respective slot to at least partially define the interior volume.

9. The macroencapsulation device of claim 1, wherein each slot is in contact with the respective projection around at least a portion of a perimeter of the respective projection to prevent fluid from flowing through the slot around the perimeter of the respective projection.

10. The macroencapsulation device of claim 1, wherein the fill port has a closed configuration in which the fill port is configured to prevent fluid from flowing through the fill port between the interior volume and an external environment.

11. The macroencapsulation device of claim 10, wherein, when the fill port is in the closed configuration, the perimeter of the interior volume is cooperatively formed by at least the fill port, the seal, and two fluid-tight closures formed between the two slots and the two projections.

12. The macroencapsulation device of claim 10, further comprising a lumen extending in a longitudinal direction through the fill port, wherein the at least one projection extends from the perimeter of the interior volume into the at least one slot in a direction substantially parallel to a length of the lumen.

13. The macroencapsulation device of claim 10, wherein the at least one projection comprises a first projection and a second projection, and wherein the at least one slot comprises a first slot and a second slot, the first projection disposed within the first slot and the second slot disposed within the second projection.

14.-20. (canceled)

21. The macroencapsulation device of claim 1, further comprising at least one population of cells disposed within the interior volume.

22. The macroencapsulation device of claim 20, wherein the at least one population of cells comprises a population of insulin-producing cells.

23. The macroencapsulation device of claim 20, wherein the fill port is in a closed configuration to encapsulate the at least one population of cells within the interior volume.

24. A method of treating a disease, the method comprising implanting the macroencapsulation device of claim 1 in a subject.

25. The method of claim 24, wherein the disease is diabetes.

26. A method of manufacturing a macroencapsulation device, the method comprising:
bonding a portion of a first membrane layer to a portion of a second membrane layer to form a seal extending around and defining at least a portion of a perimeter of an interior volume disposed between the first membrane layer and the second membrane layer;
inserting a first projection of the seal into a first slot of a frame of the macroencapsulation device; and
inserting a second projection of the seal into a second slot of the frame, the first and second slots disposed on an interior portion of the frame on opposing sides of a fill port of the frame.

27. The method of claim 26, further comprising forming a first fluid-tight closure between the first projection and the first slot, and forming a second fluid-tight closure between the second projections and the second slot.

28. The method of claim 27, wherein forming each of the first and second fluid-tight closures comprises contacting each projection with the respective slot around at least a portion of a perimeter of the respective projection to prevent fluid from flowing through the slot around the perimeter of the respective projection.

29. The method of claim 28, wherein contacting each projection with the respective slot comprises heating each of the first and second slots to a melting temperature or a glass transition temperature of the frame, and flowing material of each slot into contact with the perimeter of the respective projection.

30. The method of claim 26, further comprising sealing the fill port to prevent fluid from flowing through the fill port between the interior volume and an external environment.

31. The method of claim 26, wherein inserting each projection into the respective slot comprises placing a first flap of the first membrane layer on a first side of the frame.

32. The method of claim 31, further comprising placing a second flap of the second membrane layer on a second side of the frame opposite the first side.

33. The method of claim 32, wherein placing the first flap on the first side of the frame comprises placing the first flap on a first side of the fill port of the frame, and wherein placing the second flap on the second side of the frame comprises placing the second flap on a second side of the fill port opposite the first side of the fill port.

34. The method of claim 26, wherein inserting each seal into the respective slot comprises inserting each seal into the respective slot in a direction generally parallel to a lumen extending through the fill port.

35. The method of claim 26, wherein bonding the portion of the at least one membrane comprises at least one of heat welding and ultrasonic welding.

36. The method of claim 26, further comprising injecting at least one population of cells into the interior volume through the fill port.

37. The method of claim 36, wherein the at least one population of cells comprises a population of insulin-producing cells.

38. The method of claim 36, further comprising sealing the fill port to encapsulate the at least one population of cells in the interior volume.

39.-45. (canceled)
